(19) United States
(12) Patent Application Publication
(10) Pub. No.: US 2009/0120825 A1
(43) Pub. Date: May 14, 2009

(54) Sustainability in personal care product sales

(76) Inventors: Marcille Faye Ruman, Oshkosh, WI (US); Heidi Ellen Vanden Boom, Appleton, WI (US); Margaret Mary Posorske, Oshkosh, WI (US); Abayomi Michael Shonoiki, Combined Locks, WI (US); Louise Cynthia Ellis Coe, Appleton, WI (US); Heidi Lynn Carey, Neenah, WI (US); Jason Robert Boon, Appleton, WI (US); Heidi Bauerlein Hopkins, Neenah, WI (US); Jason Daniel Rottier, Appleton, WI (US); Christian Matthew Hirthe, Appleton, WI (US); Jane Louise Clough, Neenah, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/983,839

(22) Filed: Nov. 13, 2007

(51) Int. Cl. A61B 19/00; B65D 85/00

(52) U.S. Cl. 206/440; 206/459.5; 206/438

(57) ABSTRACT

Packaging is provided in which to supply personal care products to a consumer, wherein at least a portion of the packaging is adapted to be reused by the consumer. The packaging includes a storage bin having a storage bin bottom and a reclosable cover, and a removable bin enclosure adapted to overlie a portion of the storage bin and to be removable from the storage bin by the consumer, wherein the bin enclosure includes product-identifying graphics, and wherein the storage bin includes non-product-identifying graphics and is substantially free of product-identifying graphics. Product-identifying graphics can include product type, product size, and product count. Non-product-identifying graphics can include seasonal graphics, graphics adapted to make the storage bin appear to be a household object, or can be related to a cause.

### BACKGROUND

Personal care products such as toiletries, moist and dry wipes, diapers, training pants, incontinence products, feminine hygiene products, swim undergarments, and the like, are conventionally packaged in discardable packaging, including significant portions that are not or cannot be recycled.

Manufacturers, retailers, and consumers of personal care products recognize a need to address environmental concerns. One slogan used to promote environmental awareness and sustainability is reduce, reuse, and recycle. Current personal care product packaging schemes, however, cannot meet these goals. An opportunity exists to create extended-use packaging to help meet these goals.

### SUMMARY

The goals of reducing, recycling, and reusing can be accomplished in packaging used to deliver products to a consumer by reducing the amount of packaging for a given product, making the packaging useful and appealing enough to promote their reuse, and by using packaging materials that are recyclable. The present disclosure describes packaging that can accomplish these goals.

The present inventors undertook intensive research and development efforts concerning improving the delivery of personal care products, particularly with respect to providing packaging that can be recycled and/or reused. The packaging not only serves to contain and identify personal care products for shipping and retail purposes, but converts the packaging into another use that is tested to be highly desired by the consumer. The disclosure presented herein demonstrates personal care product packaging in the form of a reusable storage bin. After emptying the personal care product contents, a consumer can use the handy, stackable storage bins, thus extending the packaging usefulness to the consumer.

Packaging is provided in which to supply personal care products to a consumer, wherein at least a portion of the packaging is adapted to be reused by the consumer. The packaging includes a storage bin having a storage bin bottom and a reclosable cover, and a removable bin enclosure adapted to overlie a portion of the storage bin and to be removable from the storage bin by the consumer, wherein the bin enclosure includes product-identifying graphics, and wherein the storage bin includes non-product-identifying graphics and is substantially free of product-identifying graphics. Product-identifying graphics can include product type, product size, and product count. Non-product-identifying graphics can include seasonal graphics, graphics adapted to make the storage bin appear to be a household object, or can be related to a cause.

### BRIEF DESCRIPTION OF THE DRAWINGS

The foregoing and other features and aspects of the present disclosure and the manner of attaining them will become more apparent, and the disclosure itself will be better understood by reference to the following description, appended claims, and accompanying drawing.

FIG. 1 is a partially-cutaway perspective view of one aspect of a storage bin.

Repeat use of reference characters in the present specification and drawing is intended to represent the same or analogous features or elements of the present disclosure.

### DETAILED DESCRIPTION

It is to be understood by one of ordinary skill in the art that the present discussion is a description of exemplary aspects only, and is not intended as limiting the broader aspects of the present disclosure.

The present disclosure is generally directed to personal care products and how they are packaged for transport and retail sale. The personal care product can be, for instance, a moist or dry wipe, a toiletry, or an absorbent article such as a diaper, a training pant, an incontinence product, a feminine hygiene product, a medical garment, a bandage, or the like. Generally, the personal care products are disposable, meaning that they are designed to be discarded after a limited use rather than being laundered or otherwise restored for reuse.

Manufacturers, retailers, and consumers of personal care products recognize a need to address environmental concerns. One slogan used to promote environmental awareness and sustainability is reduce, reuse, and recycle. These goals can be accomplished in packaging used to deliver products to a consumer by reducing the amount of packaging for a given product, making the packaging useful and appealing enough to promote their reuse, and by using packaging materials that are recyclable. The present disclosure describes packaging that can accomplish these goals.

The packaging described herein is a value added and retailer-differentiated solution. The packaging is unique in that it includes attractive graphics and branding to convey that the storage bin is an easy-organizing extra for the customer, shopper, and/or user, and is worth a higher price per personal care product. The storage bin itself carries minimal to no information. Instead, a removable bin enclosure communicates all of the manufacturing, distribution, branding, and labeling requirements. A package violator can communicate product claims with respect to reusable storage, stylish design, and environmental sustainability.

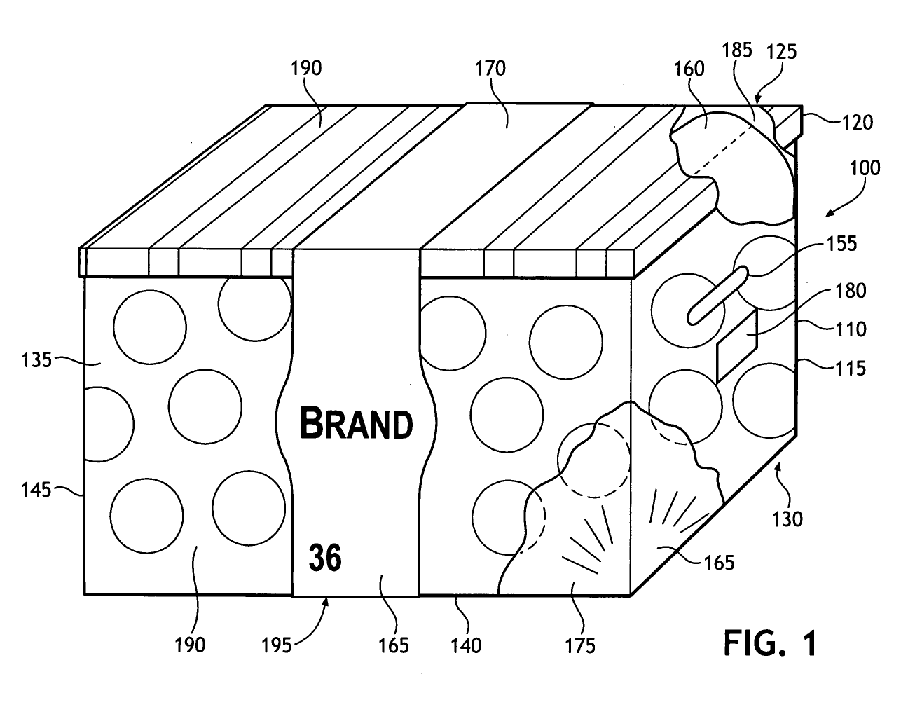

The packaging described herein is particularly useful for transporting and selling disposable personal care products including absorbent articles, wipes, toiletries, and other personal health and hygiene items. Referring to FIG. 1 for exemplary purposes, a packaging system 100 is shown. The packaging system 100 is adapted such that all or most of the packaging system 100 is readily reused and/or recycled. The packaging system 100 described herein increases the likelihood a consumer will select more environmentally sustainable packaging for disposable personal care products, thus eliminating the need for standard packaging.

As illustrated in FIG. 1, the packaging system 100 includes a storage bin 110 as its central component. The storage bin 110 includes a storage bin bottom 115 and a cover 120. The cover 120 is either partially or completely removable from the storage bin bottom 115, and is capable of being reattached to or re-set upon the storage bin bottom 115 to reclose the storage bin 110. In other aspects, the cover 120 can be in the form of a single or multiple flaps. The storage bin bottom 115 and cover 120 together define an interior space 125 of the storage bin 110.

The storage bin 110 as illustrated is generally rectilinear, but can be any suitable shape. The storage bin bottom 115 includes a lower face 130 and, if rectilinear, also includes four sides. If, for example, the storage bin 110 is generally cylindrical, the storage bin bottom 115 will include only one side 135 extending around the perimeter of the lower face 130. The storage bin 110 has a footprint, which is generally the view of the lower face 130 either from below or from the interior space 125. If the storage bin 110 is rectilinear, then the footprint will be rectilinear as well. If the storage bin 110 is cylindrical, then the footprint will be circular. The footprint can be any other suitable shape including an oval, a hemisphere, a triangle, a square, or any other shape including linear and/or arcuate portions.

The storage bin 110 also defines a horizontal perimeter 140 and a vertical perimeter 145. The horizontal perimeter 140 is generally the distance around the storage bin 110 on a horizontal plane, such as the perimeter of the lower face 130. The vertical perimeter 145 is generally the distance around the storage bin 110 on a vertical plane, such as across the cover 120, across the lower face 130, and across two opposed sides 135.

The storage bin 110 can be manufactured from any suitable material including corrugated cardboard, injection-molded or other plastic, wood, woven or nonwoven fabrics, or combinations of these. As a result, the storage bin 110 can be rigid or flexible, or a combination of these. Part or all of the storage bin 110 can be opaque, translucent, or generally transparent, and can be of any suitable color. In one example, the storage bin 110 can be manufactured from corrugate sturdy enough to support reuse of the storage bin 110 by the consumer. In another example, the storage bin 110 can be manufactured from multi-layered, insulated, liquid-resistant fabric such that a consumer can reuse the storage bin 110 as a cooler. In still another example, the storage bin 110 can be manufactured from plastic such that the consumer can reuse the storage bin 110 for semi-permanent storage, as a planter, or for any other typical household use.

Packaging used prior to this invention includes graphics targeted for point of sale and therefore do not blend with home and baby decor. Packaging of the present disclosure includes point-of-sale graphics that are removable to remove the clash between point-of-sale graphics and home decor. Finally, the storage bin 110 can feature home décor graphics, a label area, a removable cover 120, etc. Consumers want ready-to-use packaging; they do not want to assemble a storage bin 110 or a cover 120, and they want easy access to the packaging and product.

There are many storage bin styles including but not limited to, slotted boxes, telescoping boxes, folders, rigid boxes such as Bliss boxes, self-erecting boxes, corrugated common footprint containers, and interior forms. Interior forms such as liners, tubes, pads, build-ups, dividers, partitions, and other inner packaging pieces can be made in an infinite variety of ways to separate or cushion products, to strengthen the storage bin 110, or to fill voids to prevent products from moving.

In the aspects of the present disclosure in which the storage bin 110 is made from corrugate, a consumer-preferred version of a reusable storage bin 110 is a half-slotted storage bin 110 and a tray as the cover 120. Half-slotted means that the storage bin 110 has no top flaps. Interior forms may be added to the storage bin 110 to improve storage bin durability and/or to increase functionality and reusability of the storage bin 110. A Bliss box, which has no bottom flaps and which is similar to a standard shoe box, is also very appealing to consumers because it eliminates gaps and the potential for the bottom to break open.

Also, in the aspects of the present disclosure in which the storage bin 110 is made from corrugate, corrugated fiberboard, or combined board, has two main components: the linerboard and the medium. Both are made of a special kind of heavy paper called container board. Linerboard is the flat facing or liner that adheres to the medium. The medium is the corrugated or fluted paper glued between the linerboard facings. Linerboard can be single face, single wall, double wall, or triple wall. Flutes come in several common sizes or profiles ranging from A (33 flutes/foot) to F (125 flutes/foot). Smaller flute profiles provide enhanced structural and graphics capability for primary and retail packaging, while larger flute profiles deliver a greater vertical compression strength and cushioning. Different flute profiles can be combined in one piece of combined board. Mixing flute profiles allows designers to manipulate the compression strength and total thickness of the combined board.

For storage bins 110 of the present disclosure, a consumer-preferred version of a reusable storage bin 110 is a single- or double-wall structure depending on the substrates (e.g., materials, weight, flute) used. This is done to minimize the storage bin's vulnerability to crushing, abrasion, and puncturing. Consumers desire high-quality graphics on a strong and durable storage bin 110. Retail diaper and wipes boxes commonly use B or C flutes to balance aesthetic and structural requirements. In double wall containers, varied flute profiles provide advantages over flutes of the same size that are perfectly aligned. Smaller and finer fluting is desired on the outside of the storage bin to maintain high quality graphics.

Corrugating adhesive is normally bonded to the liners with a starch-based adhesive. It is available in several levels of water resistance. A flat piece of corrugated fiberboard that has been cut, slotted, and scored is called a box blank. For some box styles, in order to make a box, the two ends of the box blank must be fastened together with glue, tape, or staples. The place where these two ends meet is known as the manufacturer's joint. For storage bins 110 of the present disclosure, a glue joint on the inside of the storage bin 110 is preferred. This is done for both aesthetics and safety reasons. Also, for the storage bin 110 and cover 120 in general, it is desired to have the entire structure glued versus taped or stapled.

Accurate inside storage bin dimensions must be determined to ensure the proper fit of the products being shipped or stored. In addition, accurate outside storage bin dimensions must be determined to optimize palletizing and distributing the product. For storage bins 110 of the present disclosure, storage bin dimensions are influenced by several factors including: consumer-preferred storage bin dimensions, retailer-desired price point of offering, product quantity, product size and configuration (e.g., arrangement and orientation), pallet footprint and cube utilization, and manufacturing equipment limitations.

In other aspects of the present disclosure, windows or special cut outs can be added to the storage bin 110 to enhance functionality or aesthetic appeal, including to add air vents to the storage bin 110. In still other aspects of the present disclosure, the storage bin 110 can include a second storage bin 110 inside for strength.

The storage bin 110 as it is presented to the consumer includes disposable personal care products within the interior space 125 of the storage bin 110. Such disposable personal care products include, but are not limited to, absorbent articles, dry, moist, or wet wipes, toiletries, and other personal care items. Absorbent articles include diapers, training pants, swim pants, incontinence products, feminine hygiene products, bandages, and the like. The personal care products can be disposed within the interior space 125 in any suitable arrangement.

In another aspect of the present disclosure, the personal care products can be disposed within one or more inner packages 160. Such inner packages 160 can provide compression, containment, environmental protection, and/or organization to the personal care products. In one aspect, the inner package 160 is a flexible consumer package, which refers to non-rigid containers, such as polyethylene bags, that are adapted to contain personal care products and are adapted to be presented to a consumer. In other various aspects, the inner package 160 is manufactured from standard poly film, paper, woven or nonwoven fabric, or from any other suitable material or combination of materials and in any suitable manner. In another aspect promoting reuse of the inner package 160, the inner package 160 includes a reclosing element that allows the inner package to be reclosed after it has been opened. Such a reclosing element can be a zipper-type closure or any other suitable closure element. In another aspect, the reclosing element can make the inner package 160 airtight.

The packaging system 100 can also include a bin enclosure 165 that partially or completely envelops the storage bin 110. The bin enclosure 165 can assist in ensuring the cover 120 stays in place atop the storage bin bottom 115 through manufacturing, shipping, and retail handling. The bin enclosure 165 can also provide space on which to provide product, brand, and other labeling information. The bin enclosure 165 can also provide environmental protection to shield the storage bin 110 from dust, dirt, water, and other hazards that might otherwise adversely affect the appearance, performance, and value of the storage bin 110 and the products within.

In one aspect of the present disclosure, the bin enclosure 165 is a sleeve 170 that surrounds all or part of the vertical perimeter 145 of the storage bin 110. In general, the sleeve 170 encircles the storage bin 110 to provide both a means of keeping the cover 120 in place atop the storage bin bottom 115 and an additional surface on which to provide product, brand, and other labeling information. The sleeve 170 has a bin-facing side and an outward-facing side. The sleeve 170 can be manufactured from paperboard, poly film, woven or nonwoven fabric, or any other suitable material or combination of materials. The sleeve 170 is wrapped around the storage bin 110 and then attached to itself and/or the storage bin 110 using adhesives, tabs, or any other suitable mechanisms. The sleeve 170 can be centered on the storage bin 110 or can be offset toward one side of the storage bin 110.

In another aspect of the present disclosure, the bin enclosure 165 is an overwrap 175 that surrounds all or part of the storage bin 110. In general, the overwrap 175 encloses the storage bin 110 to provide a means of keeping the cover 120 in place atop the storage bin bottom 115; an additional surface on which to provide product, brand, and other labeling information; and environmental protection of storage bin 110 and its contents. The overwrap 175 can be manufactured from poly film, polarizing film, woven or nonwoven fabric, or any other suitable material or combination of materials. The overwrap 175 is wrapped around the storage bin 110 and then attached to itself and/or the storage bin 110 using heat, shrinking, adhesives, tabs, or any other suitable mechanisms.

In general, the bin enclosure 165, whether it is a sleeve 170 or an overwrap 175, can include indicia indicating a secondary use of the bin enclosure 165. In addition, the packaging system 100 can include both a sleeve 170 and an overwrap 175. In this aspect, the sleeve 170 is typically placed directly on the storage bin 110 as a means of keeping the cover 120 in place atop the storage bin bottom 115. The overwrap 175 is then placed to enclose both the storage bin 110 and the sleeve 170 as environmental protection of the storage bin 110 and its contents. In this aspect, either or both of the sleeve 170 and the overwrap 175 can be used as a surface on which to provide product, brand, and other labeling information.

With respect to sustainable practices, reduction is at the head of the Environmental Protection Agency's waste management hierarchy: reduce, reuse, recycle, compost and recover energy. Sustainable practices are being promoted across all segments of the packaged goods supply chain. By implementing sustainable packaging such as reusable storage bins 110, society will generate less waste, use fewer materials, and generate savings in manufacturing, transportation, storage, and disposal/recovery. Specific variables that can be controlled include greenhouse gas emissions emitted during manufacture, the environmental-friendliness of materials, product-to-package ratio, cube utilization, transportation impacts, recycled content, recovery value, renewable energy used, and sustainable innovations.

Diapers and other personal care products are already sold in corrugated boxes that consumers tend to recycle or simply discard, to end up in landfills. Also, consumers buy plastic totes to save and store baby things and other items. Shipping and retailing personal care products in a reusable storage bin 110 accrues a double benefit - recycled or discarded shipping boxes are eliminated, and the need to buy additional storage containers is eliminated as well. Consumer and manufacturer efforts are synchronized in getting personal care products to the consumer in a beautiful and earth-friendly storage bin 110.

Additionally, for the selling unit the waste is minimized and can also be made from earth-friendly materials, recycled, and/or reused. This holds true for the supplementary packaging used to protect the transportation unit (e.g., fillers, caps, corner posts, stretch wrap). Such efforts minimize materials that are put in landfills and minimize overall energy use. Further, co-packing the products employs people and avoids designing costly equipment that may have a limited lifespan due to the specialized nature of the packaging.

Transportation and warehousing are the most frequent places where damage may occur. Compression can occur with static and dynamic loads. Static loads, such as those induced by stacking in a warehouse, are affected by load, time, humidity (static and cyclic), stack pattern, and pallet type and condition. Dynamic loads, such as those induced in transit, are affected by momentary loading, clamp handling, railcar humping/coupling, and trailer sway. In addition to compression, shock (drop, impact, falls), vibration, puncture, temperature, humidity, pressure, corrosion, energy, contamination, shrink, and time are contributors to damage. Transport packaging has been historically viewed as a value retainer as opposed to a value adder. This approach is being challenged by increased contributions from transport packaging such as shelf presence, marketing, advertising, and ongoing consumer uses, including product dispensing and disposal.

For the packaging of the present disclosure, numerous precautions have been taken to ensure that the storage bin 110, which is marketed as product, arrives at the retailer in pristine condition and maintains its integrity with consumer use in the home environment. Storage bins 110 are not shipped inside of other shipping or corrugated boxes. To ensure the integrity of the storage bins 110, several variables are controlled and include double wall corrugate, shrink wrap to prevent rubbing and scuffing and to provide friction between packages while stacked, corner posts to prevent vertical crushing, top and bottom caps and slip sheets to distribute load and protect exposed edges, load securement tools in trucks, proper truck handling such as the use of forklifts only without clamping, pallet utilization above 90 percent, pallet quality such as no broken deck boards, stretch wrapping extended to the bottom of the pallet to prevent unit load shifting, limiting stacks to two-high instead of four-high, and direct shipping from co-packer to retailer distribution centers and stores to prevent additional handling.

In another aspect of the present invention, interior packaging such as another box, forms, fillers, dividers, bags, and/or cartons can be used to strengthen the storage bin 110 and further minimize susceptibility to damage in transit or use. In still another aspect of the present invention, personal care products can be shipped to a co-packer in plain packaging, which the co-packer simply slips into a storage bin 110 to minimize product handling.

A potentially key factor in making components of the packaging system 100 conducive to reuse is the design of those components. For example, a consumer is more likely to reuse the storage bin 110 if the storage bin 110 is attractive enough to complement the consumer's home decor. Features of the packaging system 100 that enhance its appeal include premium branding graphics that are of high quality and provide an abundance of “pop.” In addition, graphics can cover the front, top, and back panels of the storage bin 110, as multi-sided branding is important for bill boarding.

To add interest and thus increase the chance of reusing components of the packaging system 100, the storage bin 110 can be provided with a bin design. The bin design refers primarily to the design of the storage bin 110, including its shape, its coloring, and the graphics associated with it. The bin design can be associated with a color, a pattern, a holiday, a season, a character, a story, an action, an event, or any other suitable subject matter or combination of these. If a character, bin designs include a fictional character, a non-fictional character, and an animated character such as a cartoon character. If an event, bin designs include events such as toilet training or a specific holiday. Graphics described throughout this disclosure can include baby-related, kitchen-related, and age-related graphics, and other graphics that are attractive to a caregiver or a baby. In an example, a bin design can include a cartoon character, where the graphics placed on the personal care product relate to that cartoon character. Graphics can be depicted on the personal care product by printing, embossing, engraving, or by any other suitable means. The design can include a label area 180 to allow the consumer to label the storage bin 110.

The features selected for the storage bin 110 are selected to be representative of the reuse of the storage bin 110. Such features include the label area 180 and the cover 120, along with other features described herein. Such features ensure that the storage bin 110 functions properly during reuse and complements the user's home decor. Appropriate marriage of graphics and structure will guide the user selecting a use for the container. For example, cartoon graphics are usually more appropriate for a toddler's bedroom or playroom, while home decor graphics such as basket weave, florals, etc. are more appropriate for an adult's bedroom or the family living area, and baby-related graphics are more appropriate for a nursery.

The retail environment requires packaging to do much more than just store its contents; the package must sell the product. The graphics of the bin design, which include colors, images, and text, must be chosen with the potential end consumer constantly in mind. The selection of storage bin graphics is based on many factors including baby decor trends, home decor trends, color trends, and storage container trends.

Depending on the sophistication of printing desired, how many storage bins are needed, and the magnitude of the investment, a variety of printing processes ranging in low to high quality are available and include printing directly onto combined board, preprinting the linerboard, single-face laminating, and labeling. Specific processes include flexo postprint, CHROMAPAK color printing available from Weyerhaeuser Company, flexo advanced postprint, flexo premium postprint, flexo preprint, litho label, singleface lamination, and digital ink jet. These differ primarily in the number of colors and in line colors versus process colors versus spot colors. Corrugated graphic substrates can range from low to high end paper, including kraft, mottle white, bleached white, and clay-coated white.

In one aspect of the present disclosure, the storage bins 110 are printed using flexographic preprint. Other aspects of the present disclosure use offset lithography, digital printing, and direct printing. Printing methods can be leveraged to differentiate graphics by retailer, brand, product line, promotion, etc. In still other aspects of the present disclosure to maintain differentiation and bring innovation to the storage bin 110, 6-color offset, 150-175 line screen or higher can be used.

In other aspects of the present disclosure, further customization can be achieved with specialty decorative finishes and effects such as pearlescence, metallic inks, hot foil stamping, fluorescent inks, fragrance inks and adhesives, embossing, varnish, glitter, crackle, mirror, color change, matte, mesh, sparkling, soft-touch, flocking, lenticular, and holographics. The generous use of these special finishes or special effects can provide maximum visual “pop” and sensory appeal.

To add further interest and thus increase the chance of reusing components of the packaging system 100, the bin enclosure 165 can be provided with an enclosure design. The enclosure design refers primarily to the design of the bin enclosure 165, including its shape, its coloring, and the graphics associated with it. The enclosure design can be associated with a color, a pattern, a holiday, a season, a character, a story, an action, an event, or any other suitable subject matter or combination of these, such as those described above with respect to the bin design. For example, an enclosure design can be a cartoon character, where one or more of the graphics placed on the bin enclosure 165 and/or the shape of the bin enclosure 165 relate to that cartoon character. Graphics can be depicted on the bin enclosure 165 by printing, embossing, engraving, or by any other suitable means or combination of these.

To optimize interest and thus increase the chance of reusing the components of the packaging system 100, the bin design and the bin enclosure design can be coordinated. To be coordinated, the bin design and the bin enclosure design can be seen as related. For example, if the bin design is associated with a cartoon character including cartoon character graphics on the personal care product, then the bin enclosure design is also associated with that cartoon character, and can include one or more of cartoon character graphics and/or a shape associated with that cartoon character.

To further optimize interest and thus increase the chance of reusing the components of the packaging system 100, the inner package 160 can be provided with a package design. The package design refers primarily to the design of the inner package 160, including its shape, its coloring, and the graphics associated with it. The package design can be associated with a color, a pattern, a holiday, a season, a character, a story, an action, an event, or any other suitable subject matter. For example, the package design can include a cartoon character, where the graphics placed on the inner package relate to that cartoon character. Graphics can be depicted on the inner package by printing, embossing, engraving, or by any other suitable means or combination of these.

To spur interest in and thus increase the chance of reusing the components of the packaging system 100, the package design can be coordinated with one or both of the bin design and the bin enclosure design. To be coordinated, the package design can be seen as related to one or both of the bin design and the bin enclosure design. For example, if the bin design is associated with a cartoon character including cartoon character graphics on the personal care product, and the bin enclosure design is also associated with that cartoon character as described above, then the package design is also associated with the cartoon character and can include one or more of cartoon character graphics and a shape associated with that cartoon character.

To add further interest and thus increase the chance of reusing components of the packaging system 100, the storage bin 110 can be provided with separate cover and storage bin bottom designs. The cover and storage bin bottom designs refer primarily to the designs of the storage bin bottom 115 and cover 120, including their shapes, their coloring, and the graphics associated with them. The cover and storage bin bottom designs can be associated with a color, a pattern, a holiday, a season, a character, a story, an action, an event, or any other suitable subject matter or combination of these. For example, cover and storage bin bottom designs can include a cartoon character, where the graphics placed on the personal care product relate to that cartoon character. Graphics can be depicted on the personal care product by printing, embossing, engraving, or by any other suitable means.

The package system can also include instructions for the use of one or more of the personal care product, the storage bin 110, and the bin enclosure 165 including the sleeve 170 and the overwrap 175, or for other actions related to a user of a personal care product. The instructions can be provided with an instructions design. The instructions' design refers primarily to the design of the instructions, including their shape, their coloring, and the graphics associated with them. The instructions' design can be associated with a color, a pattern, a holiday, a season, a character, a story, an action, an event, or any other suitable subject matter. For example, the instructions' design can be a cartoon character, where the graphics placed on the instructions relate to that cartoon character. Graphics can be depicted on the instructions by printing, embossing, engraving, or by any other suitable means or combination of these.

In some cases, a certain color, pattern, etc. can be associated with a size, type, or count variant of a given personal care product. In this aspect, non-product-identifying graphics 190 on any or all of the components of the packaging system 100 can reflect or be complementary to that certain color, pattern, etc.

As used herein, the term “same as” refers to graphics or other thematic elements that depict a common character, shape, and the like. One graphic can be a different size and can have subtle differences and still be considered the same as a second graphic. For example, a large star graphic would be the same as a small star graphic because both depict a star. In another example, the graphic of a cartoon character clapping would be the same as a graphic of the cartoon character running because both depict the same cartoon character.

In another aspect of promoting reuse of the packaging system 100, the removable bin enclosure 165 includes most or all of the product-identifying graphics 195 that appear on the packaging system 100. Product-identifying graphics 195 can include the type of personal care product disposed in the interior space 125, the size or sizes of personal care products disposed in the interior space 125, and the count, amount, or volume of personal care products disposed in the interior space 125, all as applicable to the type of personal care products. Product-identifying graphics 195 can also include the manufacturer, customer service information, use instructions, patent markings, and any other information typically disposed on a package of personal care products.

In addition, the storage bin 110 itself is substantially free of product-identifying graphics 195 such that once the bin enclosure 165 is removed, the storage bin 110 is left without obvious or even detectable product-identifying graphics 195. A consumer can be more likely to reuse the storage bin 110 if the storage bin 110 is not overtly labeled with product information. Further, to enhance a consumer's interest in reusing the storage bin 110, the storage bin 110 includes non-product-identifying graphics 190 such as those described above. The non-product-identifying graphics 190 on the storage bin 110 can be registered in relation to the storage bin bottom 115. The non-product-identifying graphics 190 on the storage bin 110 can include graphical elements such as labels, hinges, hasps, locks, and other elements to enhance the look of the storage bin 110. The non-product-identifying graphics 190 can further include textured elements such as those that can be supplied by embossing.

Still further, the non-product-identifying graphics 190 can include graphical elements designed to make the storage bin 110 resemble a household item. For example, the addition of real or graphical hinges and pulls and coordinating graphics can make the storage bin 110 resemble a chest, a dresser, a cabinet, a toy box, a toy, a wardrobe, a trunk, a jewelry box, or a similar item.

In another aspect of promoting reuse of the packaging system 100, the storage bin 110 defines an interior surface 185, and the interior surface 185 includes non-product-identifying graphics 190 of the types described above.

In yet another aspect of promoting reuse of the storage bin 110, the cover 120 can include non-product-identifying graphics 190 different from and complementary with the non-product-identifying graphics 190 on the storage bin bottom 115. In addition, the non-product-identifying graphics 190 of the cover 120 can be registered in relation to the non-product-identifying graphics 190 of the storage bin bottom 115.

The attractiveness of reusing the packaging system 100 can be further enhanced by providing additional items to complement the design or use of the storage bin 110 or other component. For example, the storage bin 110 can also include writing tools such as a marker and labels so the consumer can label the contents of the storage bin 110 during reuse. In another example, the storage bin 110 can include dividers of varying types including, for example, dividers conducive to storing ornaments for storage bins 110 that are made available in conjunction with a holiday. Other inclusions can include items such as books, activities, toys, food, etc., or other items intended to inform or entertain the consumer or the child and to promote the enjoyment and reuse of the storage bin 110.

The attractiveness of reusing the packaging system 100 can be still further enhanced in a number of other ways: by providing information regarding how the consumer can obtain additional storage bins 110 without needing to purchase additional personal care products; by providing non-product-identifying graphics 190 related to a cause such as cancer research with a corresponding donation made to the cause; and by coordinating designs across product types and brands. In an example of the latter, personal care products can be sold in a storage bin 110 with non-product-identifying graphics 190 identical to or complementary with the graphics on a box of facial tissue and with the graphics on a standard box of baby wipes, thus allowing a consumer to coordinate the decoration of a nursery. Finally, packaging systems 100 of a given design can be available for only limited periods of time to enhance interest and sales. The designs of packaging systems 100 sold in different periods of time can be identical, similar, different but complementary, or completely different.

These and other modifications and variations to the present disclosure can be practiced by those of ordinary skill in the art, without departing from the spirit and scope of the present disclosure, which is more particularly set forth in the appended claims. In addition, it should be understood that aspects of the various aspects can be interchanged both in whole or in part. Furthermore, those of ordinary skill in the art will appreciate that the foregoing description is by way of example only, and is not intended to limit the disclosure so further described in such appended claims.

What is claimed is:

1. Packaging in which to supply personal care products to a consumer, wherein at least a portion of the packaging is adapted to be reused by the consumer, the packaging comprising:
a storage bin having a storage bin bottom and a reclosable cover; and
a removable bin enclosure adapted to overlie a portion of the storage bin and to be removable from the storage bin by the consumer, wherein the bin enclosure includes product-identifying graphics, and wherein the storage bin includes non-product-identifying graphics and is substantially free of product-identifying graphics.

2. The packaging of claim 1, wherein the bin enclosure is a sleeve, and further comprising an overwrap enclosing the storage bin and the sleeve.

3. The packaging of claim 1, further comprising an inner package including a plurality of personal care products.

4. The packaging of claim 1, wherein the product-identifying graphics include product type, product size, and product count.

5. The packaging of claim 1, wherein the non-product-identifying graphics include seasonal graphics.

6. The packaging of claim 1, wherein the non-product-identifying graphics include graphics adapted to make the storage bin appear to be a household object.

7. The packaging of claim 1, the storage bin further comprising an interior surface, wherein the interior surface includes non-product-identifying graphics.

8. The packaging of claim 1, wherein the cover includes non-product-identifying graphics different from and complementary with non-product-identifying graphics on the storage bin bottom.

9. The packaging of claim 1, wherein the storage bin is free of product-identifying graphics.

10. The packaging of claim 1, further comprising a textured surface.

11. The packaging of claim 1, further comprising information regarding how the consumer can obtain additional storage bins without personal care products.

12. The packaging of claim 1, wherein the packaging is made available to the consumer for a limited time of less than six weeks.

13. The packaging of claim 1, wherein the non-product-identifying graphics are related to a cause.

14. The packaging of claim 1, wherein the personal care products have a variant, wherein a color is associated with the absorbent article variant, and wherein the color is prominent in the non-product-identifying graphics.

15. The packaging of claim 1, wherein the storage bin bottom has a footprint, and wherein the footprint is a quadrilateral.

16. The packaging of claim 1, wherein the storage bin bottom has a footprint, and wherein the footprint includes an arcuate portion.

17. The packaging of claim 1, wherein the non-product-identifying graphics are registered on the storage bin bottom.

18. The packaging of claim 1, wherein the cover and the storage bin bottom each include non-product-identifying graphics, and wherein the cover non-product-identifying graphics are registered with the storage bin bottom non-product-identifying graphics.

19. A method of increasing the likelihood a consumer will select more environmentally sustainable packaging for disposable personal care products, the method comprising:
eliminating the need for standard packaging by providing the personal care products in a storage bin, the storage bin including
a storage bin bottom and a reclosable cover and defining an interior space; and
a bin enclosure adapted to overlie a portion of the storage bin and to be removable from the storage bin by the consumer, wherein the bin enclosure includes product-identifying graphics, and wherein the storage bin includes non-product-identifying graphics and is substantially free of product-identifying graphics.

20. The method of claim 19, wherein the bin enclosure includes indicia indicating a secondary use of the bin enclosure.

21. The method of claim 19, further comprising an inner package disposed in the interior space, the inner package including a plurality of disposable personal care products.
